(19) United States
(12) Patent Application Publication
(10) Pub. No.: US 2010/0160409 A1
(43) Pub. Date: Jun. 24, 2010

(54) Highly Branched Reagents For Modifying Biopharmaceuticals, Their Preparation And Use

(76) Inventors: Ralf Krahmer, Berlin (DE); Frank Leenders, Berlin (DE)

Correspondence Address: FITCH EVEN TABIN & FLANNERY, 120 SOUTH LASALLE STREET, SUITE 1600, CHICAGO, IL, 60603-3406, US

(21) Appl. No.: 11/993,425

(22) PCT Filed: Aug. 31, 2006
(86) PCT No.: PCT/EP06/08538

§ 371 Date: Dec. 20, 2007

(30) Foreign Application Priority Data
Sep. 1, 2005 (DE) 10 2005 041 570.9

(51) Int. Cl. A61K 47/48; C07C 43/13; C07C 237/04; C07C 69/34; C07D 207/36; C12N 11/02; A61K 31/4015; A61K 31/16; A61K 31/225; A61K 31/08; A61P 35/04; A61P 25/28; A61P 9/00; A61P 3/00; A61P 29/00; A61P 37/00; A61P 19/02

(52) U.S. Cl. 514/425; 568/680; 564/197; 560/190; 548/546; 435/177; 514/626; 514/547; 514/723

(57) ABSTRACT

The invention relates to compounds, which are suitable for coupling to pharmaceuticals, in particular biopharmaceuticals, in addition to conjugates from the compounds comprising biomolecules or pharmaceutically active substances. The inventive compounds are highly cross-linked and can be formed in a simple manner by using a central cross-linking module. The invention also relates to the use of conjugates as an improved formulation of pharmaceuticals and to the production thereof.

The present invention relates to compounds which are suitable for coupling to pharmaceuticals, in particular biopharmaceuticals, and to conjugates composed of the compounds and biomolecules or pharmaceutically active compounds. The compounds according to the invention are highly branched and can be readily formed by using a central branching building block. The invention also relates to the use of the conjugates as an improved formulation of pharmaceuticals, and to their preparation.

The development of biopharmaceuticals as medicaments, or for potential medicaments, and of biotechnological products for use in the field of proteomics or in the industrial field has made rapid advances in recent decades, with these advances having been crucially influenced by several factors:
a) improved isolation and purification techniques,
b) the revolutionary developments in genetic engineering and, associated with these developments, the possibility of preparing recombinant proteins,
c) the improved understanding of biochemistry and of the mechanisms of action of biopharmaceuticals, and
d) the opening up of new areas of application and methods for biotechnological products.

The efficacy and the duration of the effect of an active compound are determined by its pharmacological profile. A rapid loss of activity, which, in a general manner, is termed “clearance”, is very frequently observed in vivo in the case of biopharmaceuticals, in particular. The clearance takes place as a result of processes such as metabolism and renal excretion and as a result of the reaction of the immune system on the exogenous compound. Particularly proteinogenic active compounds, which constitute an important group of biopharmaceuticals, elicit a powerful immune response when being used therapeutically, with this response being able to lead to allergic shock. In many cases, such disadvantageous effects prevent this otherwise very advantageous class of active compounds from being used commercially or therapeutically.

Nevertheless, scientists have for many years been engaged in developing strategies for enabling biopharmaceuticals to be used therapeutically. One of the first methods was that of changing the surface charge by reacting proteins with succinic anhydride. This modification is termed succinylation (Habeeb, A.F.S.A. Arch. Biochem. Biophys. 1968, 121, 652). Covalently bonding a biologically active compound to a very wide variety of polymers constitutes one of the most successful strategies in recent years and has become one of the most important methods for improving the pharmacological and toxicological properties of biopharmaceutically active compounds. One of the polymers which is most frequently employed in this connection is the polyalkylene oxide polyethylene glycol, termed PEG for short.

Abuchowski, one of the pioneers in the field of polymer-mediated administration of biopharmaceuticals, showed that covalently coupling polyethylene glycol chains to a polypeptide molecule generates a positive pharmacological effect in the case of this active compound. The immunogenicity of a conjugate of this type is reduced, while its half-life in the blood is prolonged (U.S. Pat. No. 4,179,337, Davis et al.; Abuchowski & Davis “Enzymes as Drugs”, Holcenberg & Roberts, Eds. John Wiley & Sons, N.Y. 1981, 367-383). Furthermore, modifying biotechnological products, such as enzymes, frequently influences other biochemical parameters such as their pH stability and thermo-stability. A modification can therefore, because of an increase in thermostability, be advantageous, for example, for industrial enzymes which are to be used in washing agents and, because of an increase in pH stability, be advantageous for biopharmaceuticals with regard to the latter being administered orally.

The above-described studies greatly accelerated research in the field of the conjugation of active compounds with the polymer polyethylene glycol. The modification with polyethylene glycol also offers some advantages in the case of small conventional active compounds. The covalent bonding of a small active compound to the hydrophilic molecule polyethylene glycol increases the solubility of the conjugate and can also reduce toxicity (Kratz, F. et al. Bioorganic & Medicinal Chemistry 1999, 7, 2517-2524). The most important reviews on conjugation with polyethylene glycol are the following: Greenwald, R. B., J. of Controlled Release 2001, 74, 159-171; Zalipsky, S. Advanced Drug Delivery Reviews, 1995, 16, 157-182; Zalipsky, S. Bioconjugate Chem. 1995, 6, 150-165; N. K. Jain, N. K.; et al., Pharmazie 2002, 57, 5-29.

The chemical reaction for coupling a polyethylene glycol molecule to a biopharmaceutical requires one of the two components which are involved in the reaction to be activated. As a rule, the PEG molecule is, for this purpose, provided with a connecting molecule, i.e. what is termed the activated linker. The whole spectrum of long-established peptide chemistry is available for the activation. For the purpose of modifying amino functionalities, usually belonging to lysine residues, as building blocks of a biologically active compound, the linker is frequently activated in the form of an N-hydroxysuccinimide active ester. Harris, J. M. et al. (U.S. Pat. No. 5,672,662) developed this method for propionic acid and butyric acid linkers, while, in the case of Zalipsky, S. et al. (U.S. Pat. No. 5,122,614), an activated carbonic ester is employed. The reaction of a lysine residue with such an activated linker leads to the formation of an amide bond or urethane bond. The linking of a PEG to a biopharmaceutical is termed PEGylation, with this leading, in a number of cases, to loss of the biological activity. A reason for this can be the loss of the positive charge as a result of the formation of an amide bond at the lysine residue.

Reductive amination using PEG aldehydes represents a good alternative to that of using active esters (Harris, J. M. U.S. Pat. No. 5,252,714) because this coupling method leads to the formation of a secondary amine with the positive charge being preserved. Other coupling possibilities consist in using the maleimide method (cysteine residues) and in direct linkage, without any linker groups, when using tresyl or halogen compounds.

The most frequently employed PEGs are linear monomethoxypolyethylene glycol chains (m-PEGs). These are commercially available with molecular weights of from 1000 Da to 30 000 Da. Hitherto, there have been few commercial examples of branched m-PEG reagents. This is in spite of the fact that for various reasons it would be technically and economically expedient to use multiply branched PEG reagents:
a) As a result of the production, the quality of m-PEGs decreases with increasing molecular weight.

In particular from 30 000 Da onwards, the quality is no longer sufficient for use in the pharmaceutical industry. However, many applications require molecular weights of significantly >30 000 Da, and it is thus necessary to combine a plurality of linear m-PEGs to a branched molecule. Today this is realized, for example, using activated lysine provided with two m-PEG chains.
b) The m-PEGs commercially available today can be prepared economically only up to a molecular weight of 1000 Da in monodisperse form, i.e. having a uniform quality which can be reproduced at any time.
c) Linear chains are not constricted conformationally and can rotate freely depending on the environment. Consequently, the surface of the biopharmaceutical which is shielded by the chains is relatively small. Branched modifying reagents, which contain a plurality of PEG chains in one molecule, are being developed for the purpose of improving the surface shielding.

Hitherto, mainly amino acids are used as branching group for preparing branched polymer reagents. Glutamic acid or lysine offers the option to introduce m-PEGs at two points and then to activate the branching group at one position for coupling to the active compound. However, this process only permits the preparation of “Y-shaped” polymer reagents having one branch (Harris et al., US patent application US 2003/0114647 and U.S. Pat. No. 5,932,462; Greenwald et al., U.S. Pat. No. 6,113,906; Martinez et al., U.S. Pat. No. 5,643,575). However, owing to the fact that here, too, the bonds can rotate freely, these Y-shaped polymer reagents have only a moderately improved shielding effect compared to linear polymer reagents (Veronese, F. M. Bioconjugate Chem. 1995, 6, 62-69).

An alternative are branched polymer reagents prepared by direct coupling of two m-PEGs to a non-planar cyclic component (Yamasaki, M. et al., EP 1,270,642 A1). By virtue of the cyclic structure of the branching component, better spreading of the two m-PEG chains and thus an improved surface protection are achieved. Defrees F. describes a further method where branching can be increased even more by using a dipeptide and further by coupling to a hydrocarbon such as galactose (Defrees F. PCT application WO 2004/083258).

For the first time, Krähmer et al. have described a synthesis which allows the preparation of multiply branched polymer reagents in one step using a multi-component reaction, for example the Ugi reaction (PCT/EP2004/006135). The branching is constructed only during the synthesis and is thus not limited by using commercially available natural amino acids. The authors were able to demonstrate that branching offers considerable advantages with regard both to the intended function for active compound formulation and to the quality of the reagents. However, using this process, it is to date only possible to prepare polymer reagents having 4 chains in an economical manner, since the multicomponent reaction employed can be repeated only a limited number of times.

It has hitherto not been possible to prepare, in an economical manner, highly branched polymer reagents which may, in particular, contain more than four m-PEG chains and which are not based on using natural L-amino acids.

A further essential aspect for pharmaceutical application is the quality of polymer reagents for PEGylation. As a result of their preparation, polymers are present as a heterogeneous mixture. This is referred to as polydispersity and describes the mixture of various chain lengths and molecular weights in a polymer population. When preparing pharmaceutical products, it would, of course, be desirable to have available polymers having as low a heterogeneity as possible, in order to ensure uniformity and reproducibility of production batches. However, hitherto only a few methods for obtaining corresponding monodisperse polymer reagents suitable for pharmaceutical application have been described. Economically, using the available methods, it is only possible to prepare monodisperse m-PEGs having a molecular weight of up to 1000 Da.

As a consequence of the deficits described in the prior art, there is a large demand for highly branched modifying reagents which allow molecular weights of more than 40 kDa to be achieved, have a quality which is high and can be reproduced at any time, which exhibit an improved protective action for the modified active compound and which can be produced economically on a large scale.

Accordingly, it was an object of the invention to provide compounds which can be attached to biopharmaceuticals and which overcome the disadvantages of polymer reagents for conjugation to active compounds of the prior art at least partially.

According to the invention, this object is achieved by providing compounds of the formula (I)

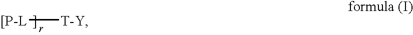

where
r is an integer between 1 and 20, preferably between 2 and 15 and more preferably between 3 and 15, and the compound of the formula (I) contains at least 3, more preferably at least 4 and even more preferably at least 5 and most preferably at least 6 groups of the formula (II), and in particular up to 50, preferably up to 30, more preferably up to 20, groups of the formula (II),
P, L, T and Y are each independently of one another a hydrocarbon radical which may contain heteroatoms, and characterized in that P exhibits at least one group of the formula (II)

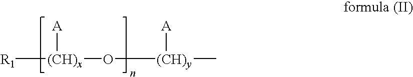

in which
A is, on each occasion independently, H, OH, C₁-C₄-alkyl, O—R₂ or CO—R₃,
R₁ is H, OH or a hydrocarbon radical which has from 1 to 50 carbon atoms and which may contain heteroatoms,
R₂ is, on each occasion independently, a hydrocarbon radical having from 1 to 6 carbon atoms,
R₃ is OH, OR₆ or NR₄R₅, in particular OH or NR₄R₅,
R₄, R₅ and R₆ are, in each case independently, H or a hydrocarbon radical which may contain heteroatoms, where R₄ and R₅ may also together form a ring system,
n is, on each occasion independently, an integer of from 2 to 1000, in particular of from 3 to 100 and preferably of from 4 to 50, and
x is, on each occasion, an integer of from 1 to 10, in particular of from 2 to 4, and
y is an integer of from 0 to 50.

According to the invention, the group T particularly preferably has the structure

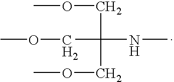

There may then be further subsequent branchings in the molecule.

The compounds according to the invention have a multiply branched skeleton which independently can be prepared by using a branching group T and/or using a branched linker L and/or a branched polymer. In preferred embodiments, T may link 1 to 20, more preferably 2 to 15 and even more preferably 3 to 15 groups of the formula P-L to a single group Y. r in formula (I) is preferably an integer ≧3, in particular ≧4, preferably ≧5 and more preferably ≧6. The compounds according to the invention are characterized in that they contain at least 3, preferably at least 4, more preferably at least 5 and most preferably at least 6 groups of the formula (II). However, according to the invention, preference is frequently also given to compounds having at least 8, in particular at least 10, groups of the formula (II).

The advantages of the highly branched and monodisperse polymer reagents according to the invention are, for example:
a) high-molecular-weight polymer reagents for pharmaceutical applications having a molecular weight of >30 000 Da can only be obtained by branching,
b) monodisperse polymer reagents having a pharmaceutically relevant molecular weight can only be obtained by branching a large number of short monodisperse starting materials,
c) only a high degree of branching of polymer reagents leads to the desired protective function with simultaneous preservation of the activity of the modified active compound and
d) unwanted crosslinking reactions can be avoided.

### Description of the Group Y

As a functional group, the compounds according to the invention contain at least one binding group Y which allows covalent attachment of the compound according to the invention to further molecules, in particular to biotechnological, pharmaceutical or synthetic active compounds, and also to surfaces or to biocatalysts. The binding group Y is preferably a compound capable of bonding covalently to a functional group present in the active compound to be coupled, for example a binding group which is capable of bonding to an amino group, a thiol group, a carboxyl group, a guanidine group, a carbonyl group, a hydroxyl group, a heterocycle, in particular with N as heteroatom (for example in histidine radicals), a C-nucleophilic group, a C-electrophilic group, a phosphate, a sulfate or the like. Also possible are non-covalent bonds, for example chelates, complexes with metals, for example at surfaces, or with radioisotopes and also bonds to silicon-containing surfaces. Suitable binding groups are, for example, a carboxylic acid or an activated carboxylic acid.

For later coupling of the compound to a biotechnological or synthetic product and also to natural compounds and industrial products, the compounds according to the invention preferably contain an activated functionality Y. In the activated form, Y is preferably selected from the group consisting of (O-alkyl)₂, —OSO₂CH₂CF₃ (tresyl), (O-aryl) azides, —CO-Q, maleimidyl, —O—CO-nitrophenyl or trichloro-phenyl, —S—S-alkyl, —S—S-aryl, —SO₂-alkenyl (vinyl-sulfone), -halogen (Cl, Br, I), where Q is independently selected from a group consisting of H, O-aryl, O-benzyl, O—N-succinimide, O—N-sulfo-succinimide, O—N-phthalimide, O—N-glutarimide, O—N-tetrahydrophthalimide, —N-norbornene-2,3-dicarboximide, hydroxybenzotriazoles and hydroxy-7-azabenzotriazoles. Preferably, Y is a group —CO-Q. A good summary of possible activations are given in the review by Zalipsky, S., published in Bioconjugate Chem. 1995, 6, 150-165.

Via group Y, the compounds according to the invention can be attached covalently to active compounds, thus forming most desirable stable conjugates.

### Description of the Groups P, L and T

The radicals P, L and T of the compound according to the invention are, in each case independently of each other, hydrogen or a hydrocarbon radical which may optionally contain heteroatoms. Here, a hydrocarbon radical is, unless explicitly stated otherwise, a radical having 1 to 100 000 carbon atoms, more preferably a radical having 1 to 10 000 carbon atoms, in some preferred cases having 1 to 50 carbon atoms, which may contain from 0 to 10 000, more preferably from 1 to 1000, heteroatoms, for example selected from the group consisting of O, P, N and S. The hydrocarbon radicals can be linear or branched and saturated or mono- or polyunsaturated. A hydrocarbon radical may also contain cyclic or aromatic portions. Preferred hydrocarbon radicals are alkyl, cycloalkyl, alkenyl, cycloalkenyl, alkynyl, cycloalkynyl, aroyl and hetero-aroyl.

On each occasion, the group P preferably contains at least one group of the formula (II). In preferred embodiments, P is branched and contains two or more groups of the formula (II).

By branching in the grouping P, compounds according to the invention are obtained which, in addition to the core branching T, may contain further subsequent branchings. These subsequent branchings may be obtained, for example, by amino acids, by a tris structure, or by a Ugi product. Preferred examples of branched groupings P are shown below.

a) On the one hand, this may be achieved by using singly branched (Y-shaped) polymer reagents in the synthesis. A particularly preferred embodiment based on a natural amino acid has the structure:

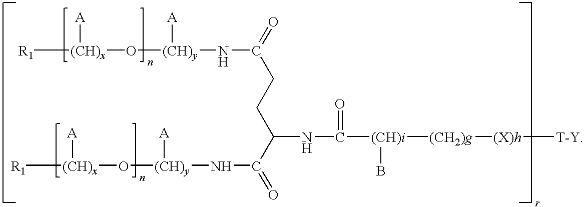

in which
A is, on each occasion independently, H, OH, C₁-C₄-alkyl, O—R₂ or CO—R₃,
R₁ is H, OH or a hydrocarbon radical which has from 1 to 50 carbon atoms, in particular up to 20 carbon atoms, and which may contain heteroatoms, in particular O, N, P or/and S,
R₂ is, on each occasion independently, a hydrocarbon radical having from 1 to 6 carbon atoms,

### R₃ is OH, OR₆ or NR₄R₅,

R₄, R₅ and R₆ are, in each case independently, H or a hydrocarbon radical which may contain heteroatoms, where R₄ and R₅ may together also form a ring system,
n is, on each occasion independently, an integer of from 2 to 1000, in particular from 3 to 100 and preferably from 4 to 50, and
x is, on each occasion, an integer of from 1 to 10, in particular of from 2 to 4, and
y is an integer of from 0 to 50, in particular from 1 to 10,
B is H, OH, C₁-C₄-alkyl, O—R₂ or CO—R₃,
i and g are in each case an integer between 0 and 20, more preferably between 1 and 10 and even more preferably between 2 and 6,

### X is N, S, P, O or C═O,

h is 0 or 1.
b) A plurality of groups of the formula (II) can be introduced in particular when, for P, a compound of the formula

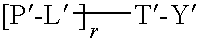

is used,
here, L′, T′, Y′ and P′ have the meanings given here for L, T, Y and P, respectively. In this case, for example, the synthesis is repeated a number of times, giving dendrimer-like structures.
c) On the other hand, this can be achieved, in particular, by using a multicomponent reaction, for example the Ugi reaction. A special embodiment is accordingly formula (III), where P═U:

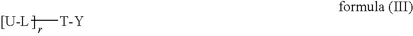

where
U is a product of the Ugi reaction of the formula (IV):

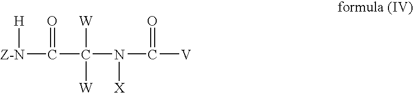

in which
the radicals V, W, X and Z are, in each case independently, a hydrocarbon radical which may contain heteroatoms, or/and V, W or/and X are hydrogen.

The preparation and the use of a group of the formula (III) or (IV) has been described in the patent application PCT/EP2004/006135.

### Description of the Group L

The group L of the compound according to the invention is preferably a linker of the form

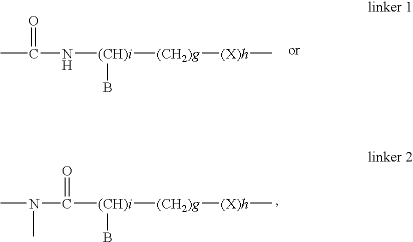

where
B is H, OH, C₁-C₄-alkyl, O—R₂ or CO—R₃,
R₁ is H, OH or a hydrocarbon radical having from 1 to 50 carbon atoms which may contain heteroatoms,
R₂ is, on each occasion independently, a hydrocarbon radical having 1 to 6 carbon atoms,

### R₃ is OH or NR₄R₅,

R₄ and R₅ are, in each case independently, H or a hydrocarbon radical which may contain heteroatoms, where R₄ and R₅ may together also form a ring system,
i and g are in each case an integer between 0 and 20, more preferably between 1 and 10 and even more preferably between 2 and 6,

### X is NR¹, S, O or C═O, and

h is 0 or 1.

However, it is also possible to use other linker structures.

If L contains a branching as described for linker 2, then, correspondingly, 2 polymers of the formula P or U may be attached, and in this case a preferred embodiment is

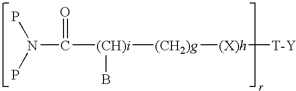

i is, on each occasion, 0 or 1,
g is, on each occasion, an integer between 0 and 10, preferably between 0 and 5,
r is an integer between 1 and 10, preferably between 1 and 3,
B is H, OH, C₁-C₄-alkyl, O—R₂ or CO—R₃,
R₁ is H, OH or a hydrocarbon radical having 1 to 50 carbon atoms which may contain heteroatoms,
R₂ is, on each occasion independently, a hydrocarbon radical having 1 to 6 carbon atoms,

### R₃ is OH or NR₄R₅,

R₄ and R₅ are, in each case independently, H or a hydrocarbon radical which may contain heteroatoms, where R₄ and R₅ may together also form a ring system,
P and P′ are, independently of one another, H or compounds which contain the group of the formula II,

### X is NR₁, S, O or C═O,

h is 0 or 1.

### Description of Group T

The branching group T is preferably represented by an alkyl chain which is branched, saturated or unsaturated and may contain heteroatoms, in particular N, S and O, for example between the branching and T. More preferably, T is an alkyl chain of the structure 1 which may attach a plurality of groups of the formula P-L to a single group of the formula Y.

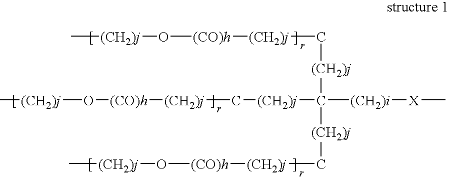

where X may be NH, C═O, S or O,
j is, on each occasion independently, an integer between 0 and 10,
r is, on each occasion independently, an integer between 0 and 3,
h is 0 or 1.

Preferred embodiments of the basic branching T have the structure

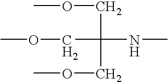

In these compounds, tris acts as a branching component.

Particular preference is given to compounds having, as at least one group T, a tris structure.

The invention provides highly branched reagents containing a plurality of and in particular a large number of groupings of the formula (II) which, in particular, confer a reduced immunogenicity, a prolonged half-life in the body, a higher proteolysis stability, an increase in solubility, a reduction in toxicity, an improved pH stability and an improved thermostability on a conjugate which is composed of a compound of the formula (I) and an active compound.

In particular, it is possible, according to the invention, to achieve good shielding using one or more short-chain groups of the formula (II), with it being possible to obtain and introduce, with good reproducibility, short-chain groups of the formula (II) (preferably with n≦50, in particular n≦25) having the same chain length. Alternatively, it is also possible to simultaneously introduce groups of the formula (II) having different chain lengths. It is furthermore also possible to employ polydisperse groups of the formula (II).

The groups of the formula (II) are preferably polyalkylene oxides, such as, for example, polyethylene glycol, polyolefin alcohols, such as, for example, polyvinyl alcohol, or polyacrylmorpholine.

In the groups of the formula (II), the radicals or variables P, R₂, R₃, R₄, R₅, n, x, y and q in a molecule or in a radical can in each case be identical or else independently of one another different. Thus, the radical of the formula (II) may, for example, be a polyalkylene oxide consisting of polyethylene oxide and polypropylene oxide groups.

For P═CO—R₃, these are polyacrylic acid groupings (R₃═OH) or polyacrylamides (R₃═NR₄R₅). Here, R₄ and R₅ may be hydrogen or a hydrocarbon radical having from 1 to 30 carbon atoms, in particular from 1 to 10 carbon atoms, more preferably from 1 to 6 carbon atoms, which may contain heteroatoms, in particular one or more heteroatoms selected from the group consisting of O, N, P and S. Together, the radicals R₄ and R₅ may also form a ring, for example a morpholine ring.

The radical R₁ is hydrogen, hydroxyl or a hydrocarbon radical having from 1 to 50 carbon atoms, more preferably from 1 to 30 carbon atoms and most preferably from 1 to 10 carbon atoms which may optionally contain heteroatoms. The radical R₁ can be saturated or mono- or polyunsaturated and linear, branched or cyclic. With particular preference, R₁ is HO, CH₃—O, CH₃—(CH₂)ₐ—O, (CH₃)₂CH—O, where a is an integer between 1 and 20. Furthermore preferably, R₁ may be selected from the group consisting of an acetal, for example (CH₃O)₂— and (CH₃—CH₂O)₂—, an aldehyde, for example OHC—CH₂—O—, an alkenyl group, for example CH₂═CH—CH₂—O—, an acrylate, for example CH₂═CH—CO₂—, or a methacrylate, for example CH₂═C(CH₃)—CO₂—, an acrylamide, for example CH₂═CH—CONH—, an aminoalkyl group, for example H₂N—CH₂—CH₂, a protected aminoalkyl group, for example A-NH—CH₂—CH₂—, where A is a protective group, in particular N-acyl, N-sulfonyl, N-silyl protective groups, such as, for example, tert-Boc-, Alloc-, Fmoc-, Tr-, Z-, Moz-, a thioalkyl group HS—CH₂—CH₂—, or a protected thioalkyl group.

In special embodiments, the radical R₁ may also be an activated functionality. In this case, R₁ is preferably selected from the group consisting of —OSO₂CH₂CF₃ (tresyl), azides, especially (O-aryl) azides, —CO-Q, maleimidyl, —O—CO-nitrophenyl or trichlorophenyl, —S—S-alkyl, —S—S-aryl, —SO₂-alkenyl (vinylsulfone), -halogen (Cl, Br, I), where Q is independently selected from a group consisting of H, O-aryl, O-benzyl, O—N-succinimide, O—N-sulfosuccinimide, O—N-phthalimide, O—N-glutarimide, O—N-tetrahydrophthalimide, —N-norbornene-2,3-dicarboximide, hydroxybenzotriazole and hydroxy-7-azabenzotriazole.

Preferably, the grouping of the formula (II) has the formula (IIa)

R₁—(CH₂—CH₂—O)ₙ—CH₂—CH₂— formula (IIa)

where n is between 0 and 1000.

On each occasion, n (as used herein, for example in, formula II or in formula IIa) is independently an integer of from 0 to 1000, more preferably from 1 to 500, even more preferably from 2 to 250, in particular at least 3 and most preferably at least 4 to 50. According to the invention, it is possible to provide compounds having a large number of groupings of the formula (II), preferably having at least 2, in particular at least 3, preferably at least 4, more preferably at least 5 and most preferably at least 9 groupings of the formula (II). However, frequently particular preference is given even to compounds which contain 3 or 4 groupings of the formula (II).

The present invention contributes to reducing the disadvantages and restrictions which have been described and which occur in the prior art. It encompasses the synthesis of multiply branched compounds which can be used for modifying natural products, industrial products, biotechnological and synthetic products or pharmaceutical active compounds.

The compounds according to the invention contain an activated linker group, which enters, within the context of a chemical reaction under mild conditions, into a covalent bond with one or more amino functionalities or other functional groups of a biotechnological or synthetic product, and at least one polymer function which influence the biochemical and pharmacological properties of the conjugate.

Preferably, the present invention provides a multiply branched structure, as well as its synthesis and use for modifying biotechnological products. To increase the degree of branching, additional branched groups of the formula P may be employed. These can be achieved, for example, by using compounds of the formula [P′-L′-]-T′- - -Y′ as P or by using products of a multicomponent reaction, for example the Ugi reaction.

The present invention preferably provides a branched polymer compound which carries only a single activated linker group, thereby avoiding crosslinking reactions. This polymer compound is hydrophilic and biologically tolerated. It is simple to prepare and opens up broad possibilities of application in connection with modifying pharmaceutical active compounds and products which are employed industrially. Conjugates of the polymer compound according to the invention and pharmaceutical active compounds enable therapeutic employment to be improved. Furthermore, by prolonging the duration of the effect, these conjugates make it possible to reduce the quantity of active compound to be administered as, for example, in the case of treating cancer diseases and infectious diseases.

In special embodiments, the polymer compound according to the invention contains a plurality of activated linker groups. In these cases, it is possible to couple a plurality of active compound molecules to the central polymer skeleton. In this manner, it is possible to achieve, for example, multivalence effects which lead to an improved biological activity of an active compound and simultaneously prolong the half-life of the molecule.

The invention furthermore relates to a process for preparing the compounds according to the invention, where, in a multistep synthesis on the basis of a branched component T, a multiply branched polymer reagent is prepared. In particular, it is possible to employ a free-radical addition of a thiol to a double bond for the synthesis. Alternatively, the Michael addition with acrylic acid derivatives may be used.

Within the context of the present invention, conjugates of the bifunctional, branched polymer compound with biologically active substances, such as proteins (for example human growth factors), enzymes, cofactors for enzymes (for example NAD+/NADH), liposomes, antibodies, small synthetic active compounds, phospholipids, lipids, nucleosides, oligonucleotides, microorganisms, human cells and surfaces are also provided.

Accordingly, the invention also relates to conjugates which comprise compounds of the formula (I) which are covalently linked to other molecules, in particular to active compounds, such as biopharmaceuticals or synthetic active compounds, or biotechnological substances which are employed in the “Life Science” field, for example in the field of proteomics or diagnostics. These substances are, for example, enzymes, in particular proteases, such as trypsin or chymotrypsin. The compounds which are linked, in the conjugates, to the compounds according to the invention are preferably biopharmaceuticals, active compounds of peptide nature or other biologically active substances. It is furthermore also possible for conjugates to be formed with surfaces or biocatalysts.

The invention furthermore relates to a pharmaceutical composition which comprises the compounds according to the invention and, in particular, the conjugates according to the invention. These pharmaceutical compositions can be employed, for example, for preventing or treating cancer, cardiovascular disorders, metabolic disorders, neuronal or cerebral disorders or inflammatory processes, such as infections, immune disorders or autoimmune disorders (for example rheumatoid arthritis).

The compounds or conjugates according to the invention are also outstandingly suitable for being used as diagnostic agents.

The invention is illustrated in more detail by the figures attached and by the examples below.

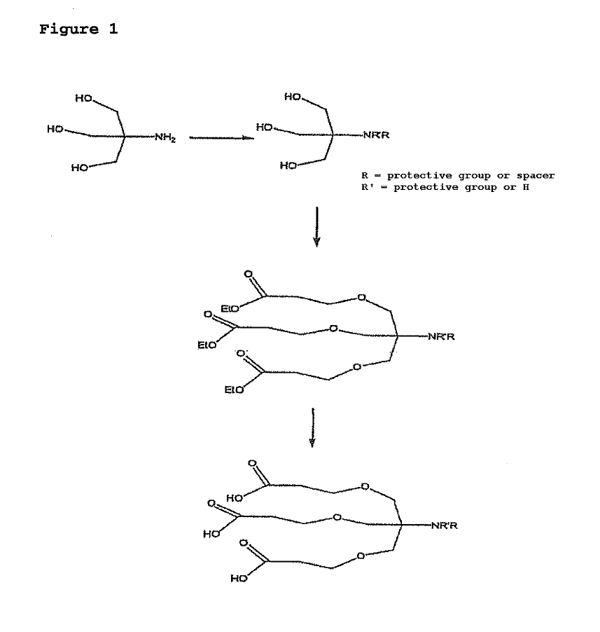

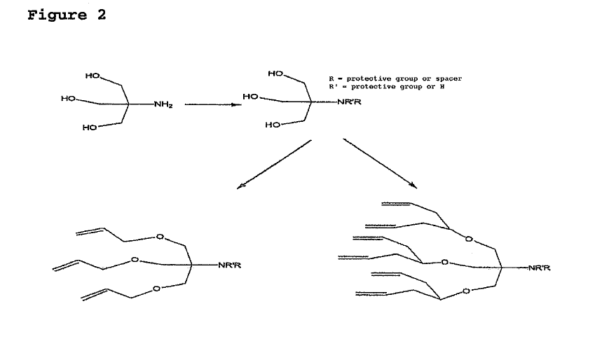

FIG. 1: Synthesis of the tricarboxylic acid in a Michael addition.

FIG. 2: Introduction of olefinic groups.

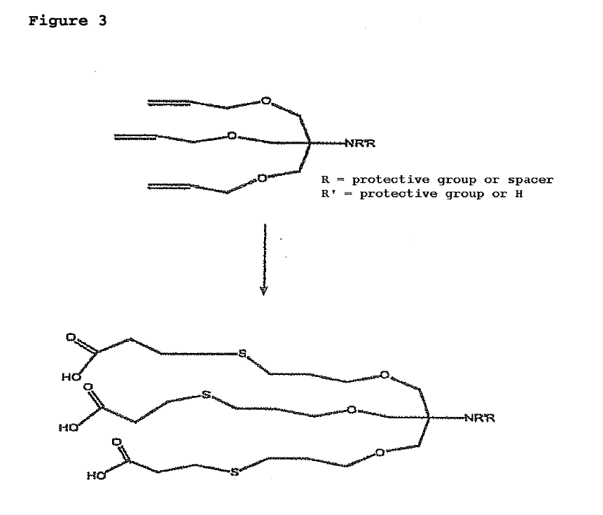

FIG. 3: Free-radical addition with formation of a thioether.

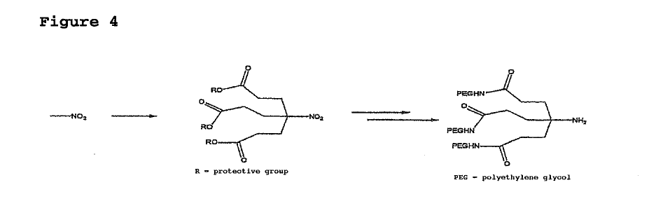

FIG. 4: Michael addition to nitromethane.

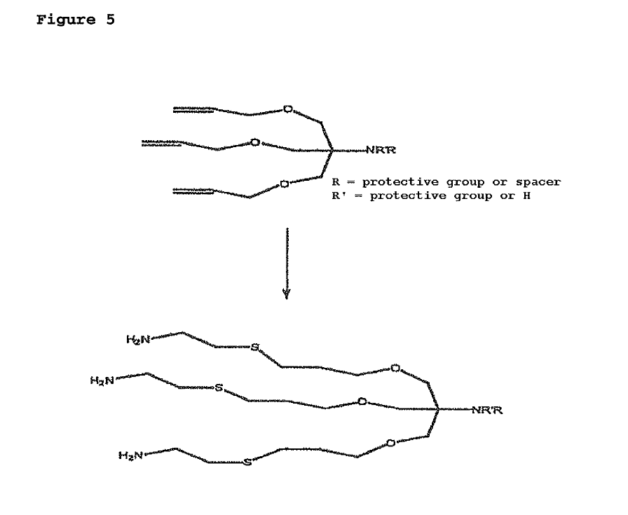

FIG. 5: Free-radical addition of 2-mercaptoethyl-amine.

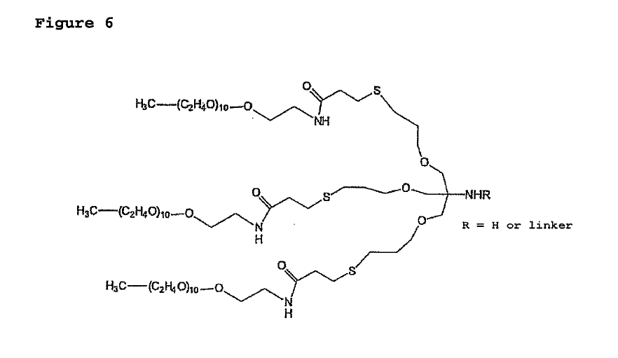

FIG. 6: Triple branching based on the central building block.

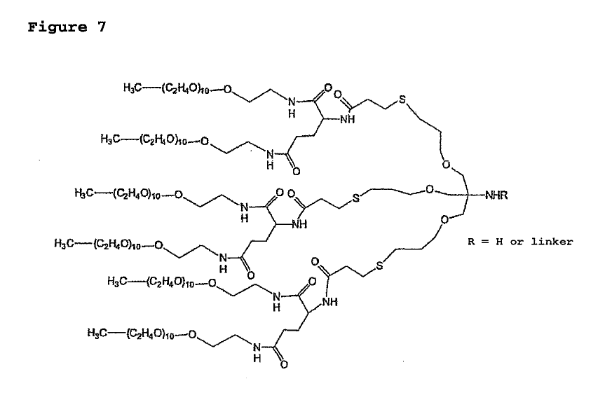

FIG. 7: Six-fold branching by incorporation of a Y-shaped PEG.

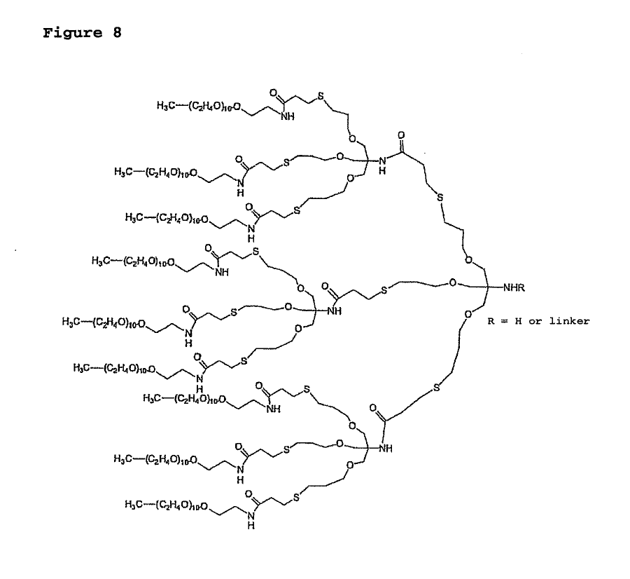

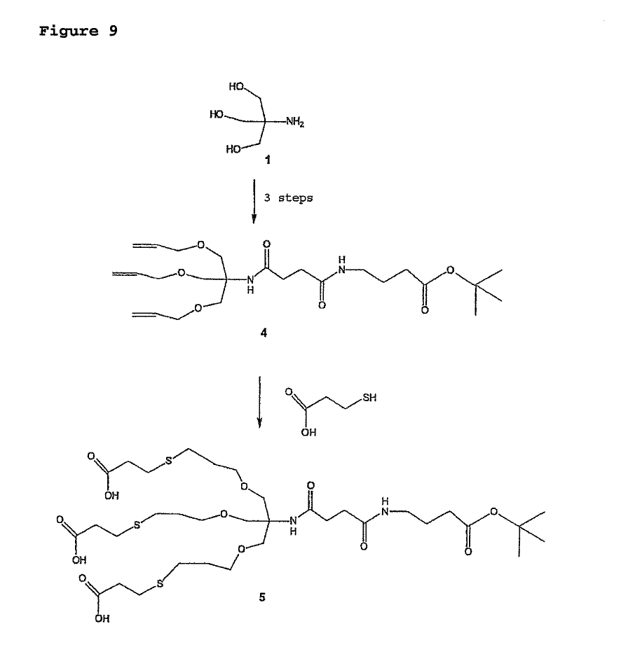

FIG. 8: Nine-fold branching by repeating the central building block.

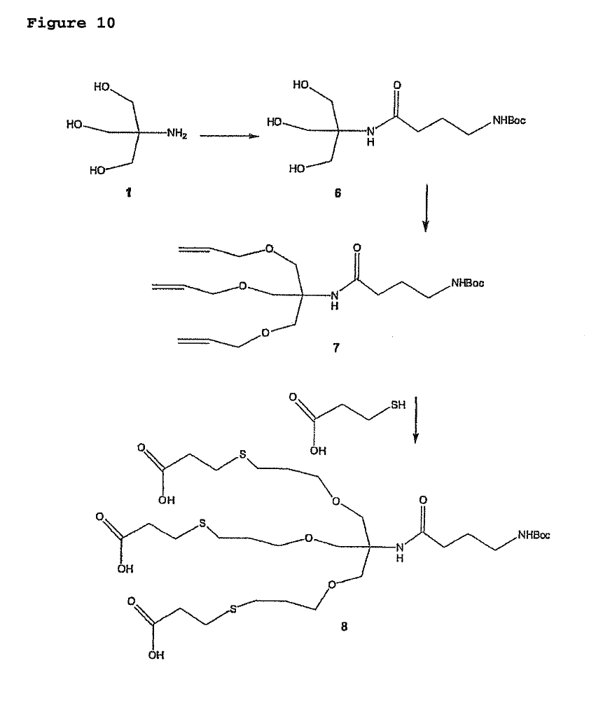

FIG. 9: Tricarboxylic acid central building block having a protected carboxylic function.

FIG. 10: Tricarboxylic acid central building block having a protected amino function.

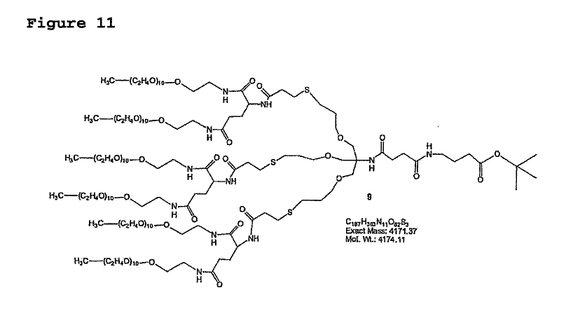

FIG. 11: 6-arm PEG having a tert-butyl-protected carboxyl function.

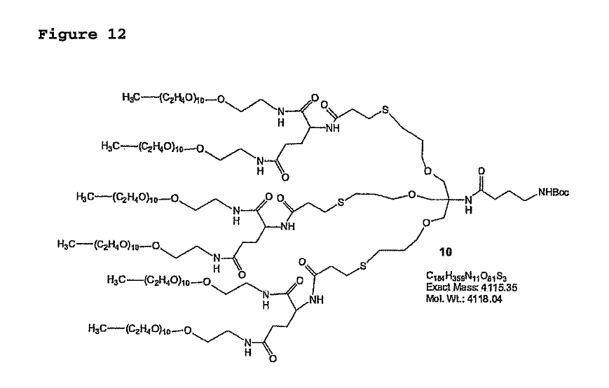

FIG. 12: 6-arm PEG having a Boc-protected amino function.

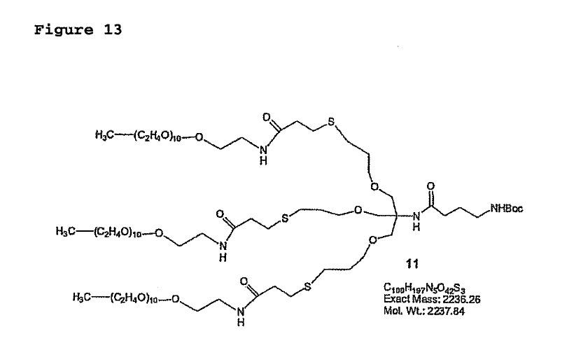

FIG. 13: 3-arm PEG having a Boc-protected amino function.

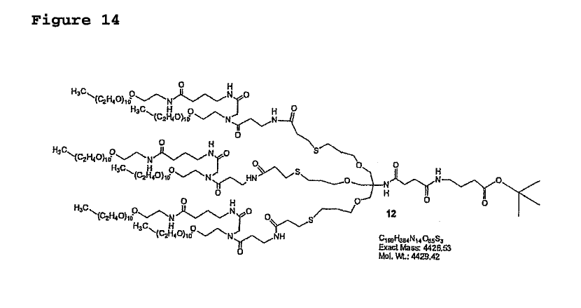

FIG. 14: 6-arm PEG having a tert-butyl-protected carboxyl function and a Ugi motive as subsequent branching.

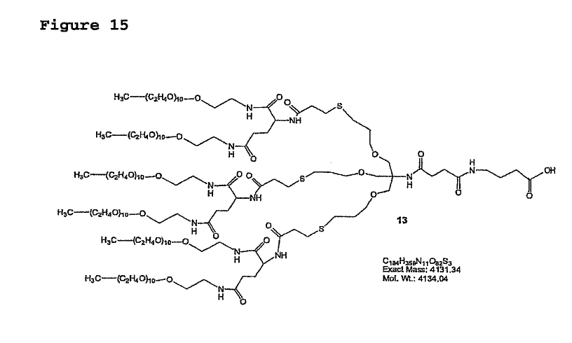

FIG. 15: 6-arm PEG having a free carboxylic acid.

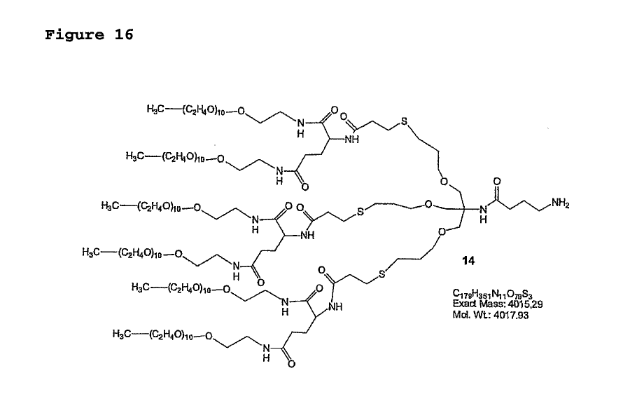

FIG. 16: 6-arm PEG having a free amine.

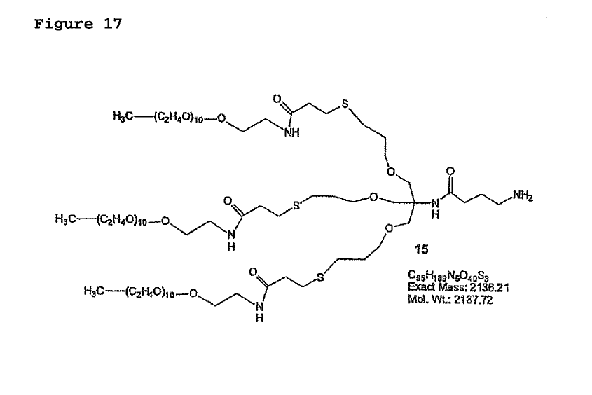

FIG. 17: 3-arm PEG having a free amine.

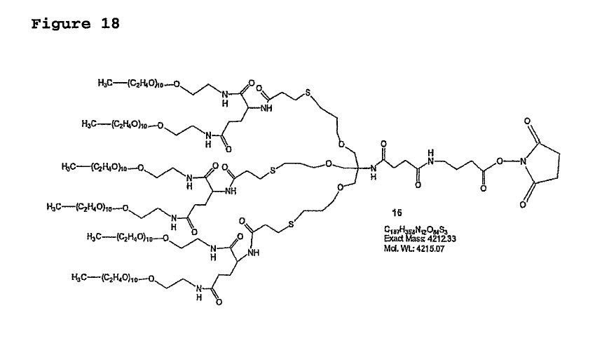

FIG. 18: 6-arm PEG.

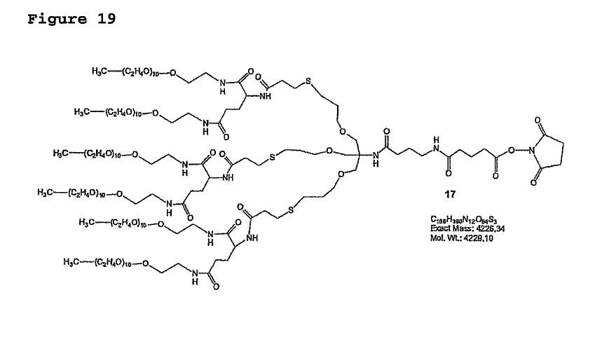

FIG. 19: 6-arm PEG having an NHS-activated glutaric acid linker.

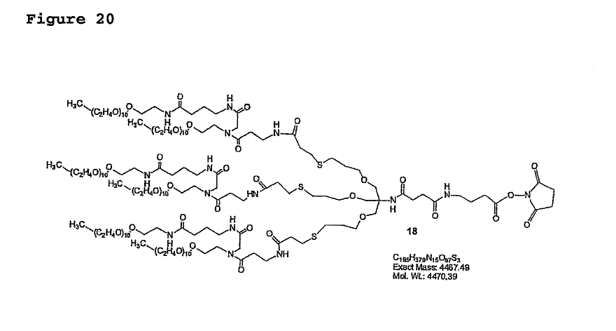

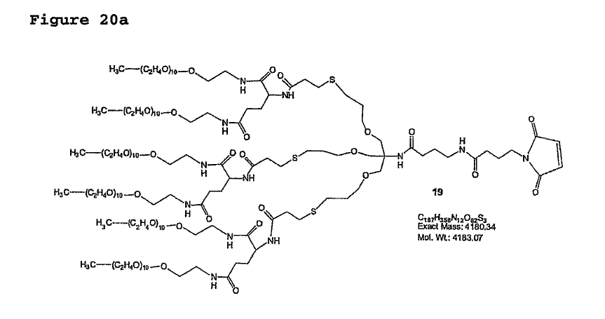

FIG. 20: 6-arm PEG NHS-activated ester with Ugi motive.

FIG. 20a: 6-arm PEG NHS-activated ester having glutaminic acid branching.

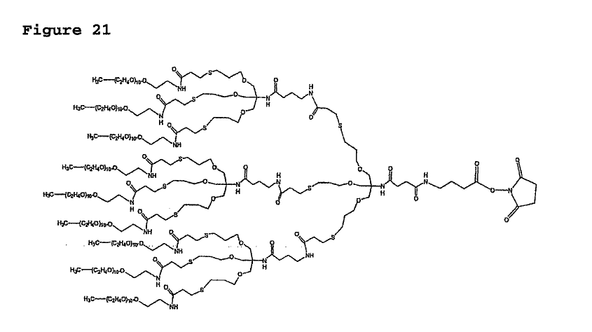

FIG. 21: 9-arm PEG; subsequent branching by using the central building block.

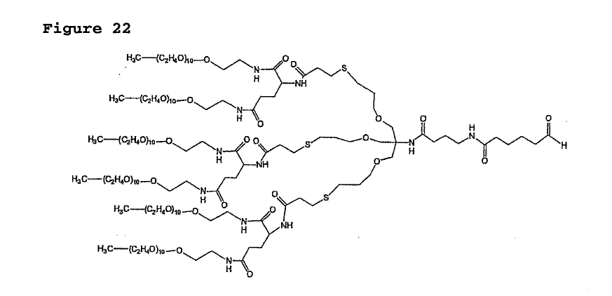

FIG. 22: Aldehyde activation for the reductive amination of amino groups.

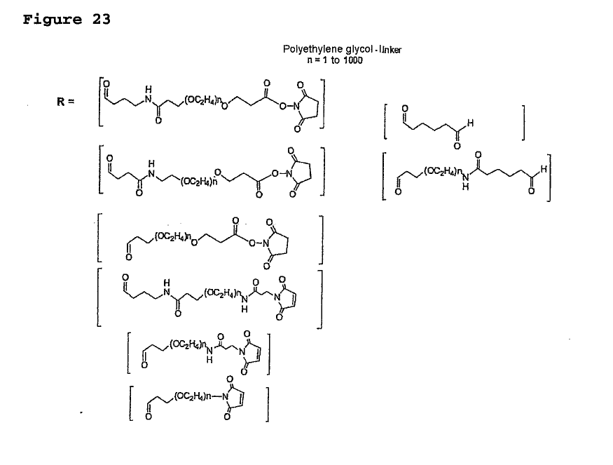

FIG. 23: Examples of binding group Y.

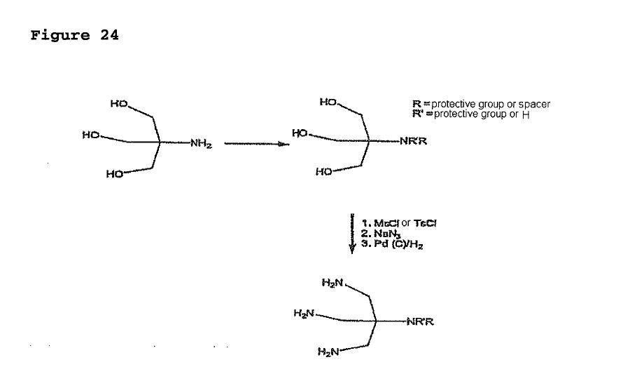

FIG. 24: Scheme showing the formation of a preferred branching component.

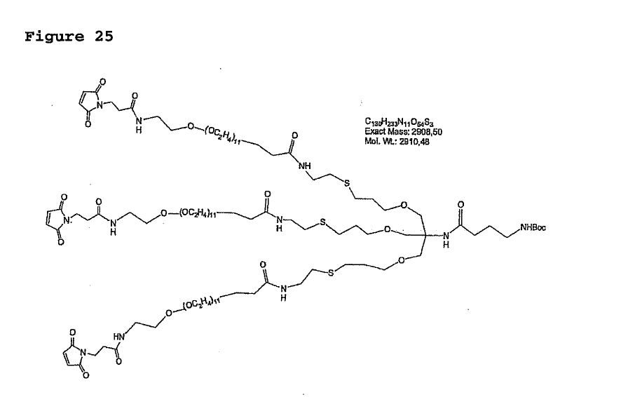

FIG. 25: 3-arm PEG having three maleimide groups, T with Boc-protected amino function.

### A. EXAMPLES OF COMPOUNDS ACCORDING TO THE INVENTION

A large number of synthetic options is available for preparing the central branching group T including the binding group Y. One option is to construct this building block from bifunctional trishydroxyamino-methane (TRIS). With regard to the economical preparation of highly branched PEGylation reagents, it is advantageous to incorporate into the central molecule functional groups which can later be reacted in good yields. Thus, one modification of the hydroxyl groups of the TRIS building block leads to a bifunctional tricarboxylic acid. However, beforehand, the amino group of TRIS is protected with a protective group (for example Boc, bis-benzyl) or a spacer.

The synthesis scheme in FIG. 1 shows the direct modification of the hydroxyl groups with an acrylic ester in the context of a Michael addition. The carboxyl function may also be introduced via a free-radical addition of a thiocarboxylic acid to an olefin or polyolefin synthesized beforehand (see FIGS. 2 and 3).

An alternative option of preparing a multifunctional tricarboxylic acid independently of TRIS as starting material uses the CH-acidic compound nitromethane. Here, nitromethane is reacted with an acrylic acid derivative in a Michael addition using a strong base. The nitro group can later be reduced to the amino function (see FIG. 4).

Starting with the triple-branched central structure, it is also possible to introduce other functional groups. Thus, the concept of the free-radical addition to olefins can also serve for introducing amino groups, as shown in FIG. 5.

Based on the various central branching building blocks, it is possible to construct highly different multiply branched PEGylation reagents. In the most simple case, linear PEG chains are coupled directly by amidation, reductive amination or free-radical addition, resulting in a triple branching. Furthermore, it is possible to generate a subsequent branching by coupling PEGs which are already branched. This may be a) the Y-shaped PEGs already known, which are prepared on the basis of natural amino acids, b) secondary amines carrying two PEG chains, branched PEGs formed in a Ugi reaction or c) the triple-branched building block is utilized repeatedly for coupling to the basic building block. These building blocks can be coupled directly or via spacers. In the three figures below, examples of these options are shown (see FIGS. 6 to 8).

### 3. Embodiments of Preferred Peg Reagent Structures:

### a) Examples of the Preparation of the Central Building Block on a Tris Basis (See FIG. 9)

### Process for Preparing 5

At 20 to 25° C., DCC (7.4 g; 36.0 mmol) is added to a solution of TRIS (3.97 g; 32.8 mmol), mono-tert-butyl succinate (5.71 g; 32.8 mmol) and NHS (0.4 g; 3.3 mmol) in DMF (100 ml). The reaction mixture is then stirred at 20 to 25° C. for 24 hours. The dicyclohexylurea formed is then removed using a filter, and the filtrate is concentrated under reduced pressure (50 mbar; 60° C.). Plug filtration through silica gel (dichloro-methane:methanol; 80:20) and subsequent crystallization from TBME gives 2 (4.5 g; 50%) as a white crystalline solid.

¹H-NMR (300 MHz, CDCl₃): δ=1.40 (9H); 2.39 (4H); 3.52 (6H); 4.70 (3H); 7.2 (1H).

¹³C-NMR (75 MHz, CDCl₃): δ=27.75; 30.50; 30.73; 60.60; 62.29; 79.59; 171.63; 172.27.

LC-MS: m/z 278.1 [M+H]⁺; C₁₂H₂₃NO₆.

At 0 to 5° C., a suspension of NaH (3.3 g; 60% in oil) in THF (30 ml) is added to a solution of 2 (4.5 g; 16.3 mmol) and allyl bromide (5.4 ml; 62 mmol) in THF (120 ml). The viscous suspension thus formed is then stirred at 0 to 5° C. for 4 hours and then at 20 to 25° C. The reaction is subsequently quenched by addition of 0.1 M HCl, and the pH is simultaneously adjusted to 6-7. The THF is then removed under reduced pressure and the residue is taken up in ethyl acetate (150 ml). This solution is washed with 0.5 M NaOH (100 ml). After phase separation, the aqueous phase is adjusted to pH 2-3 using a 1 M HCl and extracted twice with dichloromethane (in each case 100 ml). The combined organic phases are concentrated under reduced pressure. Column-chromatographic purification of the oily residue on silica gel (dichloromethane:methanol; 90:10) gives 3 (4.8 g; 85%) as a colorless viscous oil.

¹H-NMR (300 MHz, CDCl₃): δ=2.37 (4H); 3.63 (6H); 3.95 (6H); 5.05-5.35 (6H); 5.75-6.00 (3H); 7.29 (1H); 12.04 (1H).

¹³C-NMR (75 MHz, CDCl₃): δ=29.15; 30.55; 59.70; 67.86; 71.46; 116.21; 135.35; 171.29; 173.87.

C₁₇H₂₇NO₆

At 20 to 25° C., DCC (3.7 g; 17.9 mmol) is added to a solution of 3 (4.7 g; 13.8 mmol), the hydrochloride of tert-butyl 4-aminobutyrate (2.8 g; 14.3 mmol) triethylamine (2.1 ml) and NHS (0.3 mg; 2.7 mmol) in dichloromethane (100 ml). The reaction mixture is then stirred at 20 to 25° C. for 24 hours. The dicyclohexylurea is then removed using a filter, the filtrate is concentrated under reduced pressure and the residue is purified by plug filtration on silica gel. 4 (4 g; 60%) is obtained as a white solid.

¹H-NMR (200 MHz, DMSO-d6): δ=1.39 (9H); 1.56-1.62 (2H); 2.10-2.35 (6H); 3.00-3.06 (2H); 3.59 (6H); 3.91 (6H); 5.05-5.29 (6H); 5.75-5.98 (3H); 7.23 (1H); 7.80 (1H).

¹³C-NMR (50 MHz, DMSO-d6): δ=24.73; 27.75; 30.91; 31.45; 32.23; 37.78; 59.63; 67.83; 71.46; 79.48; 116.18; 135.31; 171.33; 171.71; 172.02.

LC-MS: m/z 483.6 [M+H]⁺; 505.6 [M+Na]⁺; C₂₅H₄₂N₂O₇.

3-Mercaptopropionic acid (4.4 ml; 41 mmol) is added with a syringe to a solution of 4 (2.5 g; 5.2 mmol) in toluene (45 ml). AIBN (108 mg) is added to this reaction mixture, and the resulting solution is then heated to 75 to 80° C. After a reaction time of 1.5 hours at 75 to 80° C., the reaction mixture is concentrated under reduced pressure. Chromatographic purification of the residue on silica gel (dichloromethane:isopropanol; 90:10) gives the central building block 5 (3.4 g; 80%) as a viscous colorless oil.

¹H-NMR (200 MHz, DMSO-d6): δ=1.39 (9H); 1.54-1.80 (8H); 2.12-2.35 (6H); 2.40-2.80 (18H); 2.96-3.08 (2H); 3.35-3.50 (6H); 3.54 (6H); 7.20 (1H); 7.80 (1H).

¹³C-NMR (50 MHz, DMSO-d6): δ=24.74; 26.36; 27.78; 29.22; 30.88; 31.46; 32.24; 34.46; 37.82; 59.65; 68.11; 69.17; 79.50; 171.39; 171.67; 172.05; 173.02.

LC-MS: m/z 801.7 [M+H]⁺; C₃₄H₆₀N₂O₁₃S₃.

### Process for the Preparation of 8

At 20 to 25° C., DCC (21.3 g; 103.0 mmol) is added to a solution of TRIS (11.9 g; 98.4 mmol), Boc-γ-aminobutyric acid (20 g; 98.4 mmol) and NHS (1.1 g; 9.8 mmol) in DMF (400 ml). The reaction mixture is then stirred at 20 to 25° C. for 24 hours. The reaction mixture is then filtered through a glass frit and the filtrate is concentrated under reduced pressure (50 mbar; 60° C.). The paste-like residue is then filtered through a silica gel plug (dichloro-methane:methanol; 80:20), the filtrate is reconcentrated and this residue is crystallized from ethyl acetate/methylcyclohexane (70:30). The crystallization gives 6 (24.8 g; 82%) as a white crystalline solid.

The phase-transfer catalyst tetrabutylammonium bromide (1 g) is suspended in a solution of 6 (10 g; 32.6 mmol) allyl bromide (9.1 ml; 104 mmol) and toluene (200 ml). At 20 to 25° C., NaOH (100 ml; 30%) is added to this suspension. The two-phase mixture formed is then stirred at 20 to 25° C. for 4 days. The reaction mixture is then diluted with water (150 ml). This mixture is subsequently extracted with ethyl acetate, and the organic extract is concentrated under reduced pressure. The chromatographic purification of the oily residue on silica gel (ethyl acetate:methylcyclohexane; 50:50) gives 7 (6.5 g; 46%) as a colorless viscous oil.

¹H-NMR (300 MHz, DMSO-d6): δ=1.37 (9H); 1.51-1.60 (2H); 2.0-2.08 (2H); 2.82-2.95 (2H); 3.60 (6H); 3.92 (d, 6H); 5.08-5.27 (6H); 5.75-5.92 (3H); 6.74 (t, NH, 1H); 7.17 (NH, 1H).

¹³C-NMR (75.4 MHz, DMSO-d6): δ=26.06; 28.24; 33.44; 39.32; 59.65; 67.79; 71.44; 77.40; 116.15; 135.26; 155.59; 172.23.

LC-MS: m/z 427.27 [M+H]⁺; 449.25 [M+Na]⁺; C₂₂H₃₈N₂O₆.

3-Mercaptopropionic acid (2.8 ml; 31.8 mmol) is added with a syringe to a solution of 7 (1.7 g; 4.0 mmol) in toluene (40 ml). AIBN (83 mg) is added to this reaction mixture, and the resulting solution is then heated to 75 to 80° C. The reaction mixture is then stirred at 75 to 80° C. for 1.5 hours and subsequently concentrated under reduced pressure. Chromatographic purification of the residue on silica gel (dichloromethane:isopropanol; 90:10) gives the central building block 8 (2.5 g; 82%) as a viscous colorless oil.

¹H-NMR (300 MHz, DMSO-d6): δ=1.35 (9H); 1.47-1.58 (2H); 1.63-1.78 (6H); 1.98-2.07 (2H); 2.40-2.56 (12H); 2.59-2.66 (6H); 2.83-2.92 (2H); 3.39-3.43 (6H); 3.53 (s, 6H); 6.71 (1H); 7.07 (1H); 12.15 (3H).

¹³C-NMR (75.4 MHz, DMSO-d6): δ=26.07; 26.37; 27.76; 28.26; 29.23; 33.46; 34.46; 38.77; 59.66; 68.02; 69.15; 77.43; 155.59; 172.16; 172.98.

LC-MS: m/z 745.1 [M+H]⁺; 783.1 [M+K]⁺; C₃₁H₅₆N₂O₁₂S₃.

### b) Examples of the Preparation of Highly Branched Peg Reagents

General Process for the Amidation of the Tris-Based Central Building Block with Linear or Branched Peg Amines Using the Example of 9

At 20 to 25° C., DCC (2.5 g; 12.3 mmol) is added to a solution of the tricarboxylic acid 5 (3.3 g; 4.1 mmol), the Y-shaped PEG amine (14.1 g; 12.3 mmol) and NHS (290 mg; 2.5 mmol) in dichloromethane (100 ml). The reaction mixture is then stirred at 20 to 25° C. for 24 hours. The reaction mixture is then filtered through a glass frit, and the filtrate is concentrated under reduced pressure. Without further purification steps, 9 (15.7 g; 90%) is obtained as a colorless viscous oil. If required, the product can also be purified further by column chromatography on silica gel (dichloromethane/methanol; 85:15). This general procedure applies to all other working examples.

MALDI-MS: m/z 4194.4 [M+Na]⁺; C₁₈₇H₃₆₃N₁₁O₈₂S₃.

FIG. 11: 6-arm PEG with tert-butyl-protected carboxyl function

FIGS. 12 to 14 show further working examples of the amidation:

MALDI-MS: m/z 4138.4 [M+Na]⁺; C₁₈₄H₃₅₉N₁₁O₈₁S₃ (FIG. 12)

MALDI-MS: m/z 2259.4 [M+Na]⁺; C₁₀₀H₁₉₇N₅O₄₂S₃ (FIG. 13)

MALDI-MS: m/z 4449 [M+Na]⁺; 4465.3 [M+K]⁺; C₁₉₉H₃₈₄N₁₄O₈₅S₃ (FIG. 14)

### Removal of the Protective Groups Boc and Tert-Butyl

As standard, the Boc protective group of the amino function and the tert-butyl protective group of the carboxyl function are removed using HCl (4M in dioxane) in dichloromethane. The reaction products can be purified by aqueous extraction and are used further as crude products. Yields of between 50 and 85% are obtained.

FIGS. 15 to 17 show working examples of the deblocking:

MALDI-MS: m/z 4154 [M+Na]⁺; C₁₈₄H₃₅₉N₁₁O₈₂S₃ (FIG. 15).

MALDI-MS: m/z 4016.35 [M+H]⁺; 4038.3 [M+Na]⁺; 4054.3 [M+K]⁺; C₁₇₉H₃₅₁N₁₁O₇₉S₃ (FIG. 16).

MALDI-MS: m/z 2137.3 [M+H]⁺; C₉₅H₁₈₉N₅O₄₀S₃ (FIG. 17).

### General Process for Preparing NHS-Activated Esters Using the Example of 16

At 20 to 25° C., DCC (320 mg; 1.6 mmol) is added to a solution of 13 (6.0 g; 1.5 mmol) and NHS (200 mg; 1.7 mmol) in dichloromethane. The reaction mixture is then stirred at 20 to 25° C. for 24 hours. The reaction mixture is then filtered through a glass frit, and the filtrate is concentrated under reduced pressure. Chromatographic purification on silica gel using the mobile phase dichloromethane/methanol (90:10) gives 16 (5.9 g; 90%) as a colorless oil. All other NHS-activated esters were prepared based on this process.

FIGS. 18 to 20 show working examples of NHS-activated PEG reagents:

MALDI-MS: m/z 4235.3 [M+Na]⁺; 4251.3 [M+K]⁺;

C₁₈₇H₃₅₈N₁₂O₈₄S₃ (FIG. 18).

MALDI-MS: m/z 4249.2 [M+Na]⁺; 4265.2 [M+K]⁺; C₁₈₈H₃₆₀N₁₂O₈₄S₃ (FIG. 19).

The glutaric acid linker present in compound 17 is introduced by reacting 14 with glutaric anhydride in dichloromethane. The corresponding carboxylic acid obtained in quantitative yield after aqueous work-up can be converted without further purification steps into the NHS ester.

MALDI-MS: m/z 4490.4 [M+Na]⁺; C₁₉₉H₃₇₉N₁₅O₈₇S₃ (FIG. 20).

### General Process for Preparing Peg Reagents by Maleimide Activation Using the Example of 19

At 20 to 25° C., EDCl (20 mg; 0.1 mmol) is added to a solution of 14 (0.41 g; 0.1 mmol) and NHS (5 mg) in dichloromethane (7 ml). The reaction mixture is then stirred at 20 to 25° C. for 10 hours. The reaction mixture is then diluted with dichloromethane (50 ml). The resulting solution is washed with dilute HCl (10 ml; 0.2 M). The organic phase is concentrated, giving 19 (0.43 g; quantitative) as a colorless oil.

MALDI-MS: m/z 4203.44 [M+Na]⁺; 4219.42 [M+K]⁺; C₁₈₇H₃₅₈N₁₂O₈₂S₃.

FIGS. 21 and 22 show further working examples of preferred PEG reagents.

FIG. 23 shows linker variations and activation variations.

FIG. 24 shows a scheme for forming a preferred branching component.

1. A compound of the formula (I) 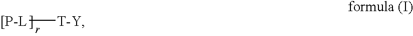
where
r is an integer between 1 and 20,
P, L, T and Y are each independently of one another a hydrocarbon radical, wherein P exhibits at least one group of the formula (IIa)
R₁—(CH₂—CH₂—O)ₙ—CH₂—CH₂— formula (IIa)
in which
R₁ is H, OH or a hydrocarbon radical which has from 1 to 50 carbon atoms,
n is, on each occasion independently, an integer of from 2 to 1000, and wherein group T exhibits the structure 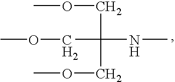
the compound containing in total at least three groups of the formula (IIa).

2. The compound as claimed in claim 1, wherein the binding group Y is selected from groups which are able to bind to an amino group, a thiol group, a carboxyl group, a guanidine group, a carbonyl group, a hydroxyl group, a heterocycle, a C-nucleophilic group, a C-electrophilic group, a phosphate or a sulfate, or are able to form a chelate or a complex with metals or are able to bond to silicon-containing surfaces.

3. The compound as claimed in claim 1 or 2, wherein the compound contains at least four groups of the formula (IIa).

4. The compound as claimed in claim 1 or 2, wherein the compound contains at least five groups of the formula (IIa).

5. The compound as claimed in claim 1 or 2, wherein the compound contains at least six groups of the formula (IIa).

6. The compound as claimed in claim 1, wherein at least one of the radicals P, L and T is branched.

7. The compound as claimed in, that claim 1, wherein the radical P contains at least two groups of the formula (IIa).

8. The compound as claimed in claim 6, wherein P is branched and, as branching component, contains an amino acid.

9. The compound as claimed in claim 8, wherein the amino acid is glutamic acid and the compound has the following structure 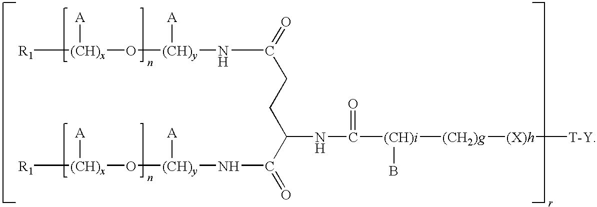
in which
r is an integer between 1 and 20,
T is as defined in claim 1,
Y is a hydrocarbon radical,
A is, on each occasion independently, H, OH, C₁-C₄-alkyl, O—R₂ or CO—R₃,
R₁ is H, OH or hydrocarbon radical which has from 1 to 50 carbon atoms,
R₂ is, on each occasion independently, a hydrocarbon radical having from 1 to 6 carbon atoms,
R₃ is OH or NR₄R₅,
R₄ and R₅ are, in each case independently, H or a hydrocarbon radical, where R₄ and R₅ may also together form a ring system,
n is, on each occasion independently, an integer of from 2 to 1000, and
x is, on each occasion, an integer of from 1 to 10,
y is an integer of from 0 to 50,
B is H, OH, C₁-C₄-alkyl, O—R₂ or CO—R₃,
i and g are each an integer between 0 and 20,
X is N, S, P, O or C═O, and
h is 0 or 1.

10. The compound as claimed in claim 1, wherein the radical P contains at least one group of the formula (P′-L′-)-T′- - -Y′,
where P′═P, L′=L, T′=T and Y′═Y.

11. The compound as claimed in claim 1, wherein the radical P is at least one group of the formula (IV) 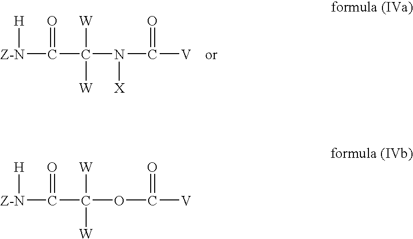
in which
the radicals V, W, X and Z are, in each case independently, a hydrocarbon radical, or/and at least on of V, W and X is a hydrogen.

12. The compound as claimed in claim 1, wherein the radical P contains at least three groups of the formula (IIa).

13. A compound of the formula (I) 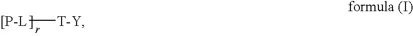
where
r is an integer between 1 and 20,
P, L, T and Y are each independently of one another a hydrocarbon radical,
wherein group L has the structure 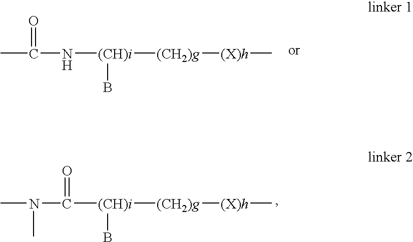
where
B is H, OH, C₁-C₄-alkyl, O—R₂ or CO—R₃,
R₁ is H, OH or a hydrocarbon radical which has from 1 to 50 carbon atoms,
R₂ is, on each occasion independently, a hydrocarbon radical having from 1 to 6 carbon atoms,
R₃ is OH or NR₄R₅,
R₄ and R₅ are, in each case independently, H or a hydrocarbon radical, where R₄ and R₅ may also together form a ring system,
r is an integer between 1 and 20,
X is NR₁, S, O or C═O, and
h is 0 or 1.

14. A conjugate which comprises a compound of the formula (I), as defined in claim 1 or 13 which is covalently bonded to a biopharmaceutical, pharmaceutical and/or synthetic active compound.

15. A compound of the formula (I), as defined in claim 1 or 13 which is covalently bonded to a surface and/or a biocatalyst.

16. A conjugate which comprises a compound of the formula (I), as defined in claim 1 or 13 which is covalently bonded to an enzyme.

17. A compound of the formula (I), as defined in claim 1 or 13, which is covalently bonded to medicinal products or adjuvants for administering active compounds.

18. A compound of the formula (I), as defined in claim 1 or 13 which is covalently bonded to tissues for hetero transplants, heart valves, liposomes or nanocapsules.

19. A pharmaceutical composition comprising a compound as claimed in claim 1 or 13.

20. A diagnostic composition comprising a compound as claimed in claim 1 or 13.

21. A method for treating cancer, cardiovascular disorders, metabolic disorders, neuronal or cerebral disorders, inflammatory processes, or immune or autoimmune disorders, the method comprising administering a pharmaceutical comprising the conjugate as claimed in claim 14.

22. A method for treating Alzheimer's disease, Parkinson's disease, infections or rheumatoid arthritis, the method comprising administering a pharmaceutical comprising the conjugate as claimed in claim 14.

23. The compound as claimed in claim 13, wherein r is an integer between 3 and 6.

24. A pharmaceutical composition which comprises a conjugate as claimed in either of claim 14.

25. A diagnostic composition which comprises a conjugate as claimed in either of claim 14.

26. A pharmaceutical composition which comprises a conjugate as claimed in either of claim 16.

27. A diagnostic composition which comprises a conjugate as claimed in either of claim 16.
